(19) United States
(12) Patent Application Publication
(10) Pub. No.: US 2023/0190857 A1
(43) Pub. Date: Jun. 22, 2023

(54) PEPTIDE WITH AMIDATED CARBOXY TERMINUS HAVING MELANOGENESIS INHIBITORY ACTIVITY AND COMPOSITION COMPRISING SAME

(71) Applicant: RUBY CROWN CO. LTD., Daegu (KR)

(72) Inventors: Yong Chool BOO, Daegu (KR); Young Mi KIM, Daegu (KR)

(73) Assignee: RUBY CROWN CO. LTD., Daegu (KR)

(21) Appl. No.: 16/758,001

(22) PCT Filed: May 24, 2019
(86) PCT No.: PCT/KR2019/006245

§ 371 Date: Apr. 21, 2020

(30) Foreign Application Priority Data
May 28, 2018 (KR) 10-2018-0060666
Sep. 18, 2018 (KR) 10-2018-0111462

(51) Int. Cl. A61K 38/00; A23L 33/17; A61K 8/64; A61P 17/00; A61Q 19/02; C07C 229/08

(52) U.S. Cl. CPC A61K 38/00; A23L 33/17; A61K 8/64; A61P 17/00; A61Q 19/02; C07C 229/08

(57) ABSTRACT

The present invention relates to a peptide with amidated carboxy terminus having melanogenesis inhibitory activity and a composition comprising same, and more specifically, to a peptide with amidated (—NH₂) carboxy terminus, composed of one amino acid sequence selected from the group consisting of SEQ ID NO: 3, SEQ ID NO: 5, SEQ ID NO: 9 to 18, and a pharmaceutical composition, cosmetic composition, and food composition for the treatment of pigmentation diseases comprising same as an active ingredient.

### TECHNICAL FIELD

This application claims the benefits of Korean Patent Applications No. 10-2018-0060666 filed on May 28, 2018 and No. 10-2018-0111462 filed on Sep. 18, 2018 with the Korean Intellectual Property Office, the disclosure of which is incorporated herein by reference in its entirety.

The present invention relates to a peptide with amidated carboxy terminus having melanogenesis inhibitory activity and a composition containing the same, and more specifically, to a peptide with amidated (—NH₂) carboxy terminus composed of one amino acid sequence selected from the group consisting of SEQ ID NO: 3, SEQ ID NO: 5 and SEQ ID NO: 9 to 18, and a pharmaceutical composition, a cosmetic composition, and a food composition for the treatment of pigmentation diseases, containing the same.

### BACKGROUND ART

Melanin is a brown- or black-colored pigment that is present in the skin, hair, eyes, and tissues, on which other pigments are deposited. Malanin makes up a large portion of a person's appearance and is also important for maintaining skin homeostasis. Melanin biosynthesis in the skin is influenced by various factors such as a hormonal change, a nutritive condition and the like. If melanin is not normally produced and its biosynthesis process is disturbed, a pigmentation defect, which is classified into hypopigmentation or hyperpigmentation, will occur. The pigmentation defect may be inherited or acquired, temporary or permanent, and may also occur in a part or all of the body. If melanin is abnormally accumulated or distributed, the melanin causes melasma, freckles, and senile lentigos, which may give an unfavorable appearance. Thus, it is a pressing issue for the cosmetics industry to prevent the undesired melanin from being accumulated.

A melanocyte (melanin-forming cell) interacts not only with an endocrine system in the body, but also with external factors such as ultraviolet rays, drugs and the like. In the skin, one melanocyte is surrounded by about 30-40 keratinocytes, and melanogenesis is regulated by a closed paracrine system. Melanin is synthesized by a chain enzymatic reaction from L-tyrosine in a melanosome, which is a cell organelle in the melanocyte. The keratinocyte stimulates the melanocyte to secrete a signaling substance which matures the melanosome and promotes melanin biosynthesis. The mature melanosome, which has accumulated melanin therein, is secreted from a dendrite of the melanocyte and transferred to a cytoplasm of an adjacent keratinocyte.

An α-melanocyte-stimulating hormone (α-MSH) is very important for melanogenesis. α-MSH is composed of an amino acid sequence of Ac-Ser-Tyr-Ser-Met-Glu-His-Phe-Arg-Trp-Gly-Lys-Pro-Val-NH₂ (SEQ ID NO: 19) and serves as an agonist of a melanocortin 1 receptor (MC1-R). When the agonist binds to MC1-R, the MC1-R activates adenylate cyclase, cAMP is increased to activate PKA, and the PKA phosphorylates a cAMP-responsive element binding protein transcription factor to finally activate a microphthalmia-associated transcription factor (MITF). MITF is also activated by Wnt, GSK3β and MAPK signaling systems and responds to various stimuli to regulate an expression level of enzymes involved in melanin biosynthesis, such as tyrosinase (TYR), tyrosinase-related protein 1 (TYRP1), and dopachrome tautomerase (DCT) (also called tyrosinase-related protein 2 (TYRP2)), etc. Agouti signaling protein is known as an antagonist of MC1-R that is competitive with α-MSH, and inhibits melanogenesis.

A peptide has drawn much attention as an active ingredient in cosmetics and cosmoceuticals due to bioactivity suitable for skin care, and various peptides have already been included as cosmetic ingredients. The amino acid sequence of the peptide is very diverse and may work in various modes of action. Further, the peptide is particularly decomposed into natural amino acids without toxicity. However, the peptide has defects in that the peptide requires high costs for peptide biosynthesis and it is inefficient for peptide to permeate the skin due to ionicity. Palmitoyl pentapeptide-4 (KTTKS), glycyl-histidyl-lysine (GHK)-Cu, acetyl hexapeptide (Argireline®) and the like alleviate a certain aspect of skin aging. Disulfanyl peptide, Angio-S (SFKLRY-NH₂) and the like have been reported as the peptide having a melanogenesis inhibitory effect.

To develop a peptide inhibitor for suppressing cellular melanogenesis, the present inventors have applied a positional scanning of synthetic peptide combinatorial library (PS-SPCL), which has been effectively used in screening various biologically active peptides. The present inventors have identified a peptide, which is expected to have melanogenesis inhibitory activity, and effects thereof by using PS-SPCL and a B16-F10 murine melanoma cell line, which is stimulated with α-MSH.

### DISCLOSURE

### Technical Problem

Accordingly, the present inventors have screened peptides composed of one to four amino acid sequences based on positional scanning of synthetic peptide combinatorial library (PS-SPCL) and have identified a novel peptide having intracellular melanogenesis inhibitory activity, thereby completing the present invention.

Thus, an object of the present invention is to provide a peptide with amidated (—NH₂) carboxy terminus composed of one amino acid sequence selected from the group consisting of SEQ ID NO: 3, SEQ ID NO: 5 and SEQ ID NO: 9 to 18.

Another object of the present invention is to provide a pharmaceutical composition for preventing or treating pigmentation diseases, containing the peptide as an active ingredient.

Further, still another object of the present invention is to provide a pharmaceutical composition for preventing or treating pigmentation diseases, consisting of the peptide as an active ingredient.

In addition, still another object of the present invention is to provide a pharmaceutical composition for preventing or treating pigmentation diseases, consisting essentially of the peptide as an active ingredient.

Still another object of the present invention is to provide a cosmetic composition containing the peptide.

Still another object of the present invention is to provide a food composition containing the peptide.

Still another object of the present invention is to provide a use of the peptide for preparing a formulation for preventing or treating pigmentation diseases.

Still another object of the present invention is to provide a method for treating pigmentation diseases, the method including: administering an effective amount of a composition containing the peptide as an active ingredient into a subject in need thereof.

### Technical Solution

In order to achieve the objects as above, the present invention provides a peptide with amidated (—NH₂) carboxy terminus composed of one amino acid sequence selected from the group consisting of SEQ ID NO: 3, SEQ ID NO: 5 and SEQ ID NO: 9 to 18.

To achieve another object of the present invention, the present invention provides a pharmaceutical composition for preventing or treating pigmentation diseases, containing the peptide as an active ingredient.

Further, the present invention provides a pharmaceutical composition for preventing or treating pigmentation diseases, consisting of the peptide as an active ingredient.

In addition, the present invention provides a pharmaceutical composition for preventing or treating pigmentation diseases, consisting essentially of the peptide as an active ingredient. To achieve still another object of the present invention, the present invention provides a cosmetic composition containing the peptide.

To achieve still another object of the present invention, the present invention provides a food composition containing the peptide.

To achieve still another object of the present invention, the present invention provides a use of the peptide for preparing a formulation for preventing or treating pigmentation diseases. To achieve still another object of the present invention, the present invention provides a method for treating pigmentation diseases, the method including: administering an effective amount of a composition containing the peptide as an active ingredient into a subject in need thereof.

Hereinafter, the present invention will be described in more detail.

The present invention provides a peptide with amidated (—NH₂) carboxy terminus composed of one amino acid sequence selected from the group consisting of SEQ ID NO: 3, SEQ ID NO: 5 and SEQ ID NO: 9 to 18.

In the present specification, the term “polypeptide” is used interchangeably with “protein” or “peptide” and, for example, refers to a polymer of amino acid residues as generally found in proteins in nature. The peptide provided in the present invention is composed of one to four amino acid sequences, and also refers to dipeptide, tripeptide and tetrapeptide in the present specification.

One letter (three letters) of amino acid used in the present specification means the following amino acids according to the standard abbreviation rules in a field of biochemistry: A (Ala): alanine; C (Cys): cysteine; D (Asp): aspartic acid; E (Glu): glutamic acid; F (Phe): phenylalanine; G (Gly): glycine; H (His): histidine; I (IIe): isoleucine; K (Lys): lysine; L (Leu): leucine; M (Met): methionine; N (Asn): asparagine; O (Ply): pyrrolysine; P (Pro): proline; Q (Gln): glutamine; R (Arg): arginine; S (Ser): serine; T (Thr): threonine; V (Val): valine; W (Trp): tryptophan; and Y (Tyr): tyrosine.

Further, the amino acid sequences of all the peptides disclosed in the present specification are described in a direction from amino terminal (or N-terminal) to carboxy terminal (or C-terminal) according to the standard abbreviation rules in a field of biochemistry. If an amino functional group (—NH₂) is represented at a carboxy terminal of the peptide described in the present specification, this case does not mean an amino terminal of amino acid sequence, but means that an amino functional group is added to a carboxy terminal through carboxy terminal amidation, which is a chemical synthesis method for peptides.

The peptide according to the present invention may be specifically composed of an amino acid sequence with amidated (—NH₂) carboxy terminus represented by SEQ ID NO: 1 (Arg-Phe-Cys-Gly-NH₂; D1 peptide), SEQ ID NO: 2 (Arg-Phe-Cys-Arg-NH₂; D2 peptide), SEQ ID NO: 3 (Arg-Phe-Trp-Gly-NH₂; D3 peptide), SEQ ID NO: 4 (Arg-Phe-Trp-Arg-NH₂; D4 peptide), SEQ ID NO: 5 (Arg-Leu-Trp-Gly-NH₂; D5 peptide), SEQ ID NO: 6 (Arg-Leu-Trp-Arg-NH₂, D6 peptide), SEQ ID NO: 7 (Arg-Leu-Cys-Gly-NH₂; D7 peptide), SEQ ID NO: 8 (Arg-Leu-Cys-Arg-NH₂; D8 peptide), SEQ ID NO: 9 (Phe-Arg-Trp-Gly; D9 peptide), SEQ ID NO: 10 (Arg-Phe-Trp-NH₂; E1 peptide), SEQ ID NO: 11 (Arg-Phe-Gly-NH₂; E2 peptide), SEQ ID NO: 12 (Arg-Leu-Gly-NH₂; E3 peptide), SEQ ID NO: 13 (Arg-Leu-Trp-NH₂; E4 peptide), SEQ ID NO: 14 (Phe-Trp-Gly-NH₂; E5 peptide), SEQ ID NO: 15 (Leu-Trp-Gly-NH₂, E6 peptide), SEQ ID NO: 16 (Arg-Trp-Gly-NH₂; E7 peptide), SEQ ID NO: 17 (Trp-Gly-NH₂; F1 peptide) and SEQ ID NO: 18 (Gly-NH₂; G1 peptide), preferably SEQ ID NO: 3, SEQ ID NO: 5 and SEQ ID NO: 9 to 18, and more preferably SEQ ID NO: 14 to 18.

As confirmed in embodiments by the present inventors, it was confirmed that all the peptides have a melanogenesis inhibitory effect, and specifically reduce a content of cellular melanin. Further, as confirmed by the present inventors, it was confirmed that the peptide inhibits melanogenesis not only in the case that B16-F10 cells are stimulated with α-MSH, but also has a melanogenesis inhibitory effect regardless of a type of melanin cells or a melanogenesis-inducing substance. In other words, the peptide also inhibits melanogenesis in human epidermal melanocytes, which is induced by various stimuli such as foskolin, theophylline, cAMP, stem cell factor, ultraviolet rays, etc. Thus, the peptide of the present invention has a feature of having melanogenesis inhibitory activity.

The peptide according to the present invention may be derived from nature, and may be synthesized by using a generally known polypeptide synthesis method (genetic engineering method and chemical synthesis). Preparation of peptides by the genetic engineering method may be performed, for example, by preparing a nucleic acid for encoding the polypeptide or functional equivalents thereof according to a conventional method. The nucleic acid may be prepared through PCR amplification by using an appropriate primer. Alternatively, DNA sequences may be synthesized according to a standard method generally known in the art, for example, by using an automatic DNA synthesizer. The prepared nucleic acid may be inserted into a vector containing at least one expression control sequence (for example, promoter, enhancer, etc.), which is operatively linked thereto and controls an expression of the nucleic acid, then may be prepared into a recombinant expression vector, then may be transformed into a host cell, and then may be cultivated in an appropriate medium and under an appropriate condition, thereby collecting a substantially pure polypeptide, which is expressed from the nucleic acid, from the culture product. The collection may be performed by using a method (for example, chromatography) generally known in the art. In this case, the “substantially pure polypeptide” means that the polypeptide according to the present invention does not contain any other proteins derived from a host cell. The genetic engineering method for polypeptide synthesis of the present invention may be the method generally known in the art. Further, the peptide according to the present invention may be easily prepared by the chemical synthesis generally known in the art. A representative method includes a liquid- or solid-phase synthesis, fragment condensation, F-MOC or T-BOC chemical method, but is not limited thereto. The present inventors have used a peptide, which is chemically synthesized by using a C-terminus amidation reaction.

Further, in case of the peptide of the present invention, not only a polypeptide having a natural amino acid sequence, but also an amino acid sequence variant thereof having a melanogenesis inhibitory effect are also included in the scope of the present invention. A variant of the polypeptide of the present invention means a peptide, in which at least one amino acid residue in the amino acid sequence of the present invention has a different sequence through deletion, insertion, non-conservative or conservative substitution, substitution of amino acid analogue or a combination thereof, while maintaining a melanogenesis inhibitory effect. An amino acid exchange, which does not change activity of molecules on the whole, is generally known in the art.

Further, in case of the peptide of the present invention, an appropriate functional group may be added thereto through phosphorylation, sulfation, acrylation, glycosylation.

The present invention provides a pharmaceutical composition for preventing or treating pigmentation diseases, containing the peptide according to the present invention as an active ingredient.

Further, the present invention provides a pharmaceutical composition for preventing or treating pigmentation diseases, consisting of the peptide according to the present invention as an active ingredient.

In addition, the present invention provides a pharmaceutical composition for preventing or treating pigmentation diseases, consisting essentially of the peptide according to the present invention as an active ingredient.

As described above, the peptide according to the present invention has very low cytotoxicity and has an excellent melanogenesis inhibitory effect, thereby providing a pharmaceutical composition for preventing or treating pigmentation diseases, containing the peptide as an active ingredient. The pharmaceutical composition according to the present invention may be used to ameliorate and alleviate a pathological condition of excessive melanin pigmentation, for example, pigmentation, melasma, freckles, blemishes, moles, spots, Ota nevus, age spots, senile lentigos, melanoderma, which are attributable to skin damage, regeneration and the like caused by a sudden change in hormone such as aging/photoaging, pregnancy, etc., wound, inflammation, burn, etc.

Thus, the pharmaceutical composition containing the peptide of the present invention may be preferably the pharmaceutical composition containing the peptide of the present invention as an active ingredient and may have a function of helping to lighten a color of melanin pigment deposited in the skin and may have a function of helping to whiten the skin by preventing a melanin pigment from being excessively deposited in the skin to inhibit an occurrence of skin pigmentation diseases.

The skin pigmentation diseases may include melasma, freckles, dark circles, lentigos, age spots, melanocytic nevus, cafe au lait spots, Ota nevus, blue nevus, hyperpigmented nevus, pigmentation of nipples, pigmentation of labia minora, blemishes and the like, but are not limited thereto.

The pharmaceutical composition according to the present invention may contain one of the peptides according to the present invention solely or may contain at least two peptides according to the present invention by selecting and mixing the peptides.

The peptide according to the present invention may be used on its own or in the form of pharmaceutically acceptable salt. In the present invention, the “pharmaceutically acceptable” means being physiologically accepted, not inhibiting an action of active ingredients when being administered into humans, and generally not causing allergic reactions or other reactions similar thereto such as gastrointestinal disturbances and dizziness. The above salt may include acid-addition salts formed by pharmaceutically acceptable free acid, and the free acid used may include organic acid and inorganic acid. The above organic acid may include citric acid, acetic acid, lactic acid, tartaric acid, maleic acid, fumaric acid, formic acid, propionic acid, oxalic acid, trifluoroacetic acid, benzoic acid, gluconic acid, methanesulfonic acid, glycolic acid, succinic acid, 4-toluenesulfonic acid, glutamic acid and aspartic acid, but is not limited thereto. Further, the above inorganic acid may include hydrochloric acid, bromic acid, sulfuric acid and phosphoric acid, preferably hydrochloric acid, but is not limited thereto.

The pharmaceutical composition according to the present invention containing the peptide having melanogenesis inhibitory activity as an active ingredient may be formulated into various dosage forms depending on an administration route by a method generally known in the art along with pharmaceutically acceptable carriers for an effect of melanin biosynthesis inhibition or whitening. The above carrier may include all the types of solvent, dispersive medium, oil-in-water or water-in-oil emulsion, aqueous composition, liposome, micro bead and microsome.

The pharmaceutical composition according to the present invention may be administered into a patient in such an amount that shows a melanogenesis inhibitory or whitening effect. For example, a general daily dosage may be administered in a range of about 0.01 to 10000 mg/kg, preferably in a range of about 1 to 100 mg/kg. The pharmaceutical composition of the present invention may be administered at one time or several times by dividing the pharmaceutical composition within a preferable range of dosage. Further, a dosage of the pharmaceutical composition according to the present invention may be appropriately selected by those skilled in the art depending on an administration route, an administration target, age, gender, weight, individual difference and disease state.

The administration route may be performed orally or parenterally. A parenteral administration method may be performed intravenously, intramuscularly, intra-arterially, intramedullary, intradurally, intracardially, percutaneously, subcutaneously, intraperitoneally, intranasally, intestinally, locally, sublingually or intrarectally, but is not limited thereto. The skin is a place to which melanogenesis mainly occurs. Thus, a percutaneous administration of the pharmaceutical composition according to the present invention may become a main route of administration, but is not limited thereto.

If being orally administered, the pharmaceutical composition of the present invention may be formulated into a dosage form such as powder, granule, tablet, pill, sugar-coated tablet, capsule, liquid medicine, gel, syrup, suspension, wafer, etc., according to a method generally known in the art along with a carrier suitable for oral administration. Examples of the suitable carrier may include sugars such as lactose, dextrose, sucrose, sorbitol, mannitol, xylitol, erythritol, maltitol and the like, starches such as maize starch, wheat starch, rice starch, potato starch and the like, celluloses such as cellulose, methyl cellulose, sodium carboxymethyl cellulose, hydroxypropyl methyl cellulose and the like, and fillers such as gelatin, polyvinyl pyrrolidone, etc. Further, in some cases, cross-linked polyvinyl pyrrolidone, agar, alginic acid, sodium alginate or the like may be added as a disintegrating agent. Furthermore, the pharmaceutical composition may further contain anti-coagulants, lubricants, humectants, perfumes, emulsifiers, antiseptics and the like.

Further, if being parenterally administered, the pharmaceutical composition of the present invention may be formulated into a dosage form of preparation for injection, preparation for percutaneous administration and preparation for nasal inhalation along with suitable parenteral carriers according to a method generally known in the art. The above preparation for injection essentially needs to be sterilized and protected from contamination with microorganisms such as bacteria and fungi. In case of the preparation for injection, the examples of suitable carrier may include solvents or dispersive media such as water, ethanol, polyol (for example, glycerol, propylene glycol, liquid polyethylene glycol and the like), a mixture thereof and/or vegetable oil, but are not limited thereto. More preferably, the suitable carrier may include Hanks' solution, Ringer's solution, PBS (phosphate buffered saline) or sterilized water for injection containing triethanolamine, isotonic solutions such as 10% ethanol, 40% propylene glycol and 5% dextrose, etc. The preparation for injection may further contain various antimicrobial agents and antifungal agents such as paraben, chlorobutanol, phenol, sorbic acid, thimerosal, etc. so as to be protected from contamination with microorganisms. Further, in most cases, the preparation for injection may further contain isotonic agents such as sugar or sodium chloride.

The preparation for percutaneous administration may include a form of ointment, cream, lotion, gel, liquid for external use, paste, liniment, aerosol, etc. In this case, the “percutaneous administration” means that an effective amount of an active ingredient contained in a pharmaceutical composition is transferred into the skin, when the pharmaceutical composition is administered into the skin. For example, the pharmaceutical composition of the present invention may be prepared into a dosage form of injection and may be administered by a method of pricking the skin on a thin injection needle with 30 gauge or directly applying to the skin. These dosage forms are described in a prescription book generally known in a field of pharmaceutical chemistry.

In case of the preparation for inhalation, a compound used according to the present invention may be conveniently transferred in the form of aerosol spray from a pressurized pack or a fog generator, by using a suitable propellant, for example, dichlorofluoromethane, trichlorofluoromethane, dichlorotetrafluoroethane, carbon dioxide or other suitable gases. In case of a pressurized aerosol, a dosage unit may be determined by providing a valve for transferring a weighed amount. For example, a gelatin capsule and cartridge used in an inhaler or insufflator may be formulated into a dosage form to contain a compound and a powder mixture of a suitable power base such as lactose or starch.

Further, the pharmaceutical composition according to the present invention may further contain at least one buffer (for example, saline solution or PBS), carbohydrate (for example, glucose, mannose, sucrose or dextran), antioxidant, bacteriostatic agent, chelant (for example, EDTA or glutathion), adjuvant (for example, aluminum hydroxide), suspending agent, thickening agent and/or preservative.

Further, the pharmaceutical composition of the present invention may be formulated into a dosage form by using a method generally known in the art so as to provide a fast, suspended or prolonged release of an active ingredient after being administered into a mammal.

In addition, the pharmaceutical composition of the present invention may be administered alone or in combination with a generally known material (for example, compound) having a melanogenesis inhibitory or whitening effect.

The present invention provides a cosmetic composition containing at least one of the peptides according to the present invention.

The peptide according to the present invention has an excellent effect of inhibiting melanogenesis and has very low cytotoxicity, thereby providing a pharmaceutical composition containing at least one of the peptides according to the present invention.

The cosmetic composition according to the present invention may contain the peptide according to the present invention solely or may contain at least two peptides according to the present invention by selecting and mixing the peptides.

The cosmetic composition according to the present invention may be preferably used without limitation in a desired region for skin whitening as the cosmetic composition containing at least one of the peptides according to the present invention as an active ingredient, and may be used in a region to which pigmentation occurs, for example, face, nipple, areola, external genital, abdominal wall midline, navel, armpit, elbow, knee, etc.

In particular, a distribution of melanin pigments in nipples or external genitals differs depending on women, and a color in those regions is changed from pink to black, etc. depending on an amount of melanin pigments distributed therein. Thus, the peptide having melanogenesis inhibitory activity according to the present invention may be used for women who suffer from stress due to the color of the above regions.

The cosmetic composition of the present invention may be prepared into any dosage form, which is conventionally prepared in the art. In addition to the peptide according to the present invention, the cosmetic composition may contain a dermatologically acceptable medium or base, and thus may be prepared in the form of adjuvant which is conventionally used in a field of cosmetic science and may be locally or systemically applied.

Further, in addition to the peptide according to the present invention, the cosmetic composition of the present invention may further contain fat materials, organic solvents, dissolvents, thickening and gelling agents, softeners, antioxidants, suspending agents, stabilizers, foaming agents, flavoring agents, surfactants, water, ion type or non-ion type emulsifiers, fillers, sequestering and chelating agents, preservatives, vitamins, blocking agents, humectants, essential oils, dyes, pigments, hydrophilic or lipophilic activators, lipid vesicles, or adjuvants, which are conventionally used in a field of cosmetic science or dermatology, such as any other ingredients conventionally used in cosmetics. And, the above ingredients may be introduced in such an amount that is generally used in the field of dermatology.

A suitable dosage form of cosmetic composition may be provided, for example, in the form of solution, gel, solid or paste anhydrous product, emulsion obtained by dispersing an oil phase in a water phase, suspension, microemulsion, microcapsule, micro-granulocyte, or ion type (liposome) or non-ion type vesicle dispersing agent, or in the form of cream, toner, lotion, powder, ointment, spray or conceal stick. Further, the dosage form of cosmetic composition may be prepared in the form of foam or aerosol composition further containing a compressed propellant. Products, to which the cosmetic composition of the present invention may be added, may include a dosage form of skin lotion, skin softener, skin toner, astringent lotion, emollient toilet water, nourishing toilet water, astringent, lotion, milky lotion, moisture lotion, nourishing lotion, body cream, massage cream, nourishing cream, moisture cream, hand cream, essence, nourishing essence, pack, soap, shampoo, cleansing foam, cleansing lotion, cleansing cream, body lotion, body cleanser, treatment, beauty liquid, emulsion, pressed powder, loose powder, eye shadow, etc., but are not limited thereto.

A content of the peptide of the present invention, contained in the cosmetic composition of the present invention, may be in a range of 0.00001 to 100 wt %, preferably 0.001 to 10 wt % with regard to the total weight of the cosmetic composition, and may be appropriately determined by those skilled in the art considering factors such as a desired whitening effect, a degree of application, a type of dosage form, stability of the peptide in the cosmetic composition, etc.

The present invention provides a food composition containing at least one of the peptides according to the present invention.

The food composition according to the present invention, containing at least one of the peptides having melanogenesis inhibitory activity, may include all the forms of functional food, nutritional supplement, health functional food and food additives and the like. The above types may be prepared in various forms according to a conventional method generally known in the art. The food composition of the present invention may be prepared in the form of powder or concentrate so as to be used in the form of food additives.

Meanwhile, the food composition according to the present invention may contain the peptide according to the present invention solely or may contain at least two peptides according to the present invention by selecting and mixing the peptides. The food composition according to the present invention may be preferably a food composition containing at least one of the peptides according to the present invention as an active ingredient.

Also, the food composition according to the present invention may be a food composition for whitening.

The above “health functional food” means a food prepared and processed by using a raw material or ingredient, which has functionality useful for the human body according to No. 6727 of the Health Functional Food Act, and the “functionality” means taking the food for the purpose of adjusting nutrients with regard to structures and functions of the human body or obtaining an effect valuable for hygiene uses such as a physiological action, etc.

For example, as the health food, the food composition according to the present invention itself may be prepared into the form of tea, juice and drink so as to be drunk, or may be subject to granulation, encapsulation and pulverization to be taken in. Further, the food composition of the present invention may be prepared in the form of composition by being mixed with generally known materials or active ingredients, which are generally known to have a melanogenesis inhibitory or whitening effect.

The food composition of the present invention may contain conventional food additives, and whether a certain item is suitable as the “food additives” or not is decided on the basis of specifications and standards on the item according to the general rules, general testing methods and the like of the Food Additives Code approved by the Ministry of Food and Drug Safety, unless there are other regulations.

The items listed on the “Food Additives Code” may include, for example, chemically synthesized compounds such as ketones, glycine, potassium citrate, nicotinic acid, cinnamic acid, etc., natural additives such as persimmon color, licorice extract, crystalline cellulose, kaoliang color, guar gum, etc., and mixed formulations such as L-sodium glutamate formulation, alkali additives for noodles, preservative formulation, tar color formulation, etc.

Further, the present invention provides food additives containing the peptide, which are used in food for preservatives, disinfectants, antioxidants, spices, seasoning agents, sweeteners, congeners, swelling agents, fortifying agents, improving agents, emulsifiers, various nutrients, flavoring agents such as synthetic and natural flavoring agents, color additives, color fixing agents, enhancers (cheese, chocolate, etc.), pectic acid and salts thereof, alginic acid or salts thereof, organic acid, protective colloidal thickeners, pH adjusting agents, stabilizers, antifoaming agents, solvents, releasing agents, antiseptics, quality-improving agents, glycerin, alcohol, carbonators, etc., used in carbonated beverages, or other additives for preparing food, or used as an essential raw material of food material (supplementary raw material). In this case, the food additives may be added in the above food by being immersed, sprayed or mixed into the food.

Further, the functional food may be prepared by adding the food composition of the present invention into beverages (including alcoholic beverages), fruits and processed foods thereof (for example, canned fruits, bottled foods, jam, marmalade, etc.), fishes, meats and processed foods thereof (for example, ham, sausage, corned beef, etc.), breads and noodles (for example, udon, buckwheat noodles, instant noodles, spaghetti, macaroni, etc.), fruit juice, various kinds of drink, cookies, taffy, dairy products (for example, butter, cheese, etc.), edible vegetable fats and oils, margarine, vegetable protein, retort foods, frozen foods, various kinds of seasoning agent (for example, soybean paste, soy source, source, etc.).

A content of the food composition containing the peptide according to the present invention may be preferably in the range of 0.00001 to 100 wt %, and more preferably 0.001 to 10 wt % in the finally prepared food, but is not limited thereto. The present invention provides a use of at least one peptide with amidated (—NH₂) carboxy terminus for preparing a formulation for preventing or treating pigmentation diseases, selected from the group consisting of SEQ ID NO: 3, SEQ ID NO: 5 and SEQ ID NO: 9 to 18.

The present invention provides a method for treating pigmentation diseases, the method including: administering an effective amount of a composition containing at least one peptide with amidated (—NH₂) carboxy terminus selected from the group consisting of SEQ ID NO: 3, SEQ ID NO: 5 and SEQ ID NO: 9 to 18 into a subject in need thereof.

In the present invention, the “effective amount” means an amount of showing an effect of ameliorating, treating, preventing, detecting and diagnosing pigmentation diseases, or inhibiting or reducing pigmentation diseases when being administered into an individual, and the “individual” may refer to animals including mammals, in particular, humans, and also refer to cells, tissues, organs, etc., derived from the animals. The subject may include a patient who needs the above effect.

In the present invention, the “treatment” inclusively refers to ameliorating pigmentation diseases or symptoms thereof, which may include healing or substantially preventing pigmentation diseases, or ameliorating conditions, and may include alleviating, healing or preventing one symptom or most symptoms caused by pigmentation diseases, but is not limited thereto.

In the present invention, the term “comprising ˜” is used the same as “containing” or “including” and does not exclude an additional ingredient, element, method, step or the like, which is not mentioned with regard to the composition or the method. The term “consisting of ˜” means excluding an additional element, step, ingredient or the like, which is not described otherwise. The term “consisting essentially of” means including an ingredient, element or step, which is described with regard to the scope of composition or method, as well as an ingredient, element, step or the like, which does not substantially affect basic properties thereof.

### Advantageous Effects

Thus, the present invention provides a novel peptide having melanogenesis inhibitory activity and a pharmaceutical, cosmetic or food composition containing the same as an active ingredient. The peptide according to the present invention has an effect of very effectively inhibiting melanogenesis while not damaging cell viability.

### DESCRIPTION OF THE DRAWINGS

FIG. 1a is a view showing results of positional scanning of synthetic peptide combinatorial library (PS-SPCL) with regard to intracellular melanogenesis. B16-F10 cells, which are a murine melanoma cell line, were stimulated with a vehicle control group or a corresponding peptide pool, after which a content of melanin was measured. Each panel shows results obtained from a tetrapeptide pool, of which amino acid was identified, and a sequence of X and O in the upper part of the panel indicates positional properties of amino acid of the tetrapeptide pool. Each of positions O is determined as one of 20 L-amino acids and the rest of five Xs include a mixture of 20 L-amino acids. FIG. 1b is a view showing an effect on B16-F10 cell viability as test results of MTT assay for each peptide combination. [FIG. 1a: representing a p value compared to cells stimulated with α-MSH only, and FIG. 1b: representing a p value compared to control group]

FIG. 2a is a view showing experimental results representing an effect of each tetrapeptide on cellular melanin biosynthesis. A cell-based assay of FIG. 2a was performed by treating B16-F10 cells with a vehicle or a peptide at a certain concentration, then stimulating the resultant cells with 100 nM of α-MSH, then measuring absorbance at 475 nm in 72 hours later, and then measuring a content of melanin. The amino acid sequences described in FIG. 2a are all described in a direction from amino terminus to carboxy terminus. An amino functional group (—NH₂) represented at a peptide terminus represents an amino functional group added by amidation of a carboxy terminus, which is used in peptide synthesis. FIG. 2b is a view showing an effect of each tetrapeptide on B16-F10 cell viability as test results of MTT assay for each tetrapeptide. [FIG. 2a: representing a p value compared to cells stimulated with α-MSH only, and FIG. 2b: representing a p value compared to control group]

FIG. 3a is a view showing experimental results representing an effect of D3, D5 and D9 tetrapeptides on a melanin content of B16-F10 cells. FIG. 3a shows a melanin content of cells which are treated with a peptide at a concentration of 10-30 μM and stimulated with 100 nM of α-MSH for 72 hours. The melanin content was normalized with regard to a total protein content. FIG. 3b is a view showing an effect of each tetrapeptide on B16-F10 cell viability as test results of MTT assay for D3, D5 and D9 tetrapeptides. In FIGS. 3a and 3b, coumaric acid and arbutin were used as a positive control group. [FIG. 3a: representing a p value compared to cells stimulated with α-MSH only, and FIG. 3b: representing a p value compared to control group]FIG. 4a shows an effect of peptides, in which a sequence of two or three amino acids is defined. A cell-based assay of FIG. 4a was performed by treating B16-F10 cells with a vehicle or a peptide at a certain concentration, then stimulating the resultant cells with 100 nM of α-MSH, then measuring absorbance at 475 nm in 72 hours later, and then measuring a content of melanin. FIG. 4b is a view showing an effect of each peptide on B16-F10 cell viability as test results of MTT assay for peptides, in which a sequence of two or three amino acids is defined. [FIG. 4a: representing a p value compared to cells stimulated with α-MSH only, and FIG. 4b: representing a p value compared to control group]

FIG. 5a is a view showing experimental results representing an effect of glycine derivatives on a melanin content of B16-F10 cells. FIG. 5a shows a melanin content of cells which are treated with glycine derivatives at various concentrations and stimulated with 100 nM of α-MSH for 72 hours. The melanin content was normalized with regard to a total protein content. FIG. 5b is a view showing an effect of each peptide on B16-F10 cell viability as test results of MTT assay for peptides, in which a sequence of glycine derivatives is defined. In FIGS. 5a and 5b, coumaric acid and arbutin were used as a positive control group. [FIG. 5a: representing a p value compared to cells stimulated with α-MSH only, and FIG. 5b: representing a p value compared to control group]

FIG. 6a is a view showing experimental results representing an effect of tetrapeptide D3, tripeptide E5, dipeptide F1 and monopeptide G1 on a content of extracellular and intracellular melanins in HEMs cell lines stimulated with α-MSH. FIG. 6b is a view showing an effect of each peptide on HEMs cell viability as test results of MTT assay for HEMs cell lines. [FIG. 6a: representing a p value compared to cells stimulated with α-MSH only, and FIG. 6b: representing a p value compared to control group]

FIG. 7a is a view showing an effect on B16-F10 cell viability as test results of MTT assay for glycinamide-HCl. FIG. 7b is a view showing experimental results representing an effect of glycinamide-HCl on a melanin content of B16-F10 cells. [FIG. 7a: representing a p value compared to control group, and FIG. 7b: representing a p value compared to cells stimulated with α-MSH only]

FIG. 8a is a view showing an effect of glycinamide-HCl on activity of tyrosinase (TYR) with regard to B16-F10 cell lines stimulated with α-MSH. FIG. 8b shows a protein expression level of tyrosinase (TYR), tyrosinase-related protein 1 (TYRP1), dopachrometautomerase (DCT) and β-actin. [representing a p value compared to cells stimulated with α-MSH only]

FIGS. 9a and 9b show an effect of glycinamide-HCl on a phosphorylation level of cAMP-responsive element binding protein (CREB) and extracellular signal-regulated kinase (ERK), and a protein level of microphthalmia-associated transcription factor (MITF) with regard to B16-F10 cell lines stimulated with α-MSH. [FIG. 9a: representing a p value compared to groups, and FIG. 9b: representing a p value compared cells stimulated with α-MSH only]

FIG. 10 shows peptide sequences having the melanogenesis inhibitory activity identified in the present invention.

### MODE FOR INVENTION

Hereinafter, the present invention will be described in more detail.

However, the following Examples are provided only for the purpose of illustrating the present invention, and thus the content of the present invention is not limited thereto.

### Experimental Method

1. Peptide Synthesis

A synthetic tetrapeptide combinatorial library package was purchased from Peptron (Daejeon, the Republic of Korea). A library includes four positional sub-libraries, that is, OXXX—NH₂, XOXX—NH₂, XXOX—NH₂, and XXXO—NH₂. In this case, each of positions O includes one of 20 L-amino acids, and a position X includes an equimolar mixture of 20 L-amino acids. Peptides of the library were synthesized through a C-terminus amidation reaction.

Additionally, a peptide pool and each peptide were prepared by using a peptide custom-made service of Peptron Co. (Daejeon, Korea). In an example, a peptide, which was chemically synthesized by using a carboxy terminus amidation, was used. A type or purity of peptide was confirmed by using mass spectrometry (MS) and high performance liquid chromatography (HPLC).

2. Cell Culture

Cells were cultured in a humidified incubator at 37° C. and 5% CO₂. Murine melanoma B16-F10 cell lines were cultured in Dulbecco' Modified Eagle Medium containing 10% fetal bovine serum and antibiotics (100 U/mL of penicillin, 0.1 mg/mL of streptomycin and 0.25 μg/mL of amphotericin B), which were purchased from the American Type Culture Collection (Manassas, Va., USA). A human epidermal melanocyte (HEM), derived from a pigmented human neonatal foreskin, was purchased from Cascade Biologics (Portland, Oreg., USA) and cultured in medium 254 containing human melanocyte growth supplements (Cascade Biologics) and antibiotics.

3. Cell Viability

Cell viability was measured by using a 3-(4,5-dimethylthiazol-2-yl)-2,5-diphenyltetrazolium bromide (MTT) assay. B16-F10 cell lines were treated with test peptides at various concentrations for 72 hours. The cells were washed with PBS and cultured in 100 μl of culture fluid, to which 1 mg/mL of MTT (Amresco, Solon, Ohio, USA) was added, for three hours. After that, the culture fluid was removed therefrom and formazan accumulated inside the cells was extracted with 100 μl of dimethyl sulfoxide (DMSO). Absorbance of the extracted solution was measured at 595 nm by using SPECTROstar Nano microplate reader (BMG LABTECH GmbH, Ortenberg, Germany).

4. Melanogenesis Inhibitory Activity

A melanin formation inhibitory effect of the peptide to be analyzed was confirmed by using B16-F10 cell lines and HEM. The B16-F10 cells were treated with test peptides at various concentrations and stimulated for 72 hours by using 100 nM of α-MSH. Hexapeptide B6 (Phe-Ser-His-His-Leu-Gly-NH₂) (SEQ ID NO: 20), p-coumaric acid and arbutin were used as a control group. A level of extracellular melanin was measured by using a conditioned medium in which cells were previously cultured. Intracellular melanin was extracted at 60° C. for 60 minutes by using 1.0 M NaOH. A content of melanin was spectroscopically measured by measuring absorbance at 475 nm and resultant values were normalized with regard to a total protein content of cells by using Bio-Rad DC assay.

5. TYR Activity Assay

B16-F10 cells were treated with glycinamide-HCl for 60 minutes and stimulated for 24 hours by using 100 nM of α-MSH. The cells were dissolved in 10 mM of cold Tris-HCl buffer solution (pH 7.4) containing 120 mM of sodium chloride, 25 mM of potassium chloride, 2.0 mM of ethylene glycol tetraacetic acid, 1.0 mM of ethylenediaminetetraacetic acid, 0.5% triton X-100, protease inhibitor cocktail (Roche, Mannheim, Germany). Cytolysate was centrifuged at 13,000×g at 4° C. for 15 minutes to obtain a cell-free extract therefrom. TYR activity was measured by using L-tyrosine and L-3,4-dihydroxyphenylalanine. A reaction mixture (200 μL) includes 100 mM of sodium phosphate (pH 6.8), 1.0 mM of L-tyrosine, 42 μM of L-3,4-dihydroxy phenylalanine and a cell-free extract (40 μg of protein), which was cultured at 37° C. A change in absorbance was measured at 475 nm by using Spectrostar Nano microplate reader.

6. Western Blotting

Western blotting was performed as described in a document [Study on phenolic compounds and novel peptides inhibiting UVB- and PM10-induced MMP1 expression and melanogenesis in skin cells. PhD Thesis 2017. Kyungpook National University, Korea]. B16-F10 cells were dissolved in 10 mM of cold Tris-HCl buffer solution (pH 7.2) containing 150 mM of NaCl, 5 mM of EDTA, 0.1% sodium dodecyl sulfate (SDS), 1% triton X-100, 1% deoxycholate, 1 mM of phenylmethylsulphonyl fluoride and protease inhibitor cocktail (Roche). A part of cytolysate (30 μg of protein) was mixed with Laemmli sample buffer, then heated at 95° C. for five minutes to be denaturalized, and then was subject to electrophoresis in 10% SDS-polyacrylamide gel. After the electrophoresis, protein was transferred from gel to polyvinylidene difluoride membrane (Amersham Pharmacia, Little Chalfont, UK). Primary antibodies for TYR, TYRP1, DCT, MITF and @-actin were purchased from Santa Cruz Biotechnology (Santa Cruz, Calif., USA). Primary antibodies for CREB, phospho-CREB (Ser133), extracellular signal-regulated kinase (ERK) and phospho-ERK (Thr202/Tyr204) were purchased from Cell Signaling (located in Danvers, Mass., USA). The membrane was cultured overnight at 4° C. along with the primary antibodies and cultured at the room temperature for one hour along with secondary antibodies (Cell Signaling) bound with horseradish peroxidase. A protein band was visualized by using picoEPD Western Reagent kit (Elpis-Biotech, Daejeon, South Korea), and a depth of image was analyzed by using National Institutes of Health ImageJ program.

7. Statistical Analysis

Data are indicated as mean±SD of three independent experiments. Experimental results were statistically analyzed through one-way ANOVA by using SigmaStat v.3.11 software (Systat Software Inc., San Jose, Calif., USA), and then all the experimental groups were compared with a single control group through Dunnett test. If a p value was less than 0.05, the value itself was indicated in a graph.

### Example 1: Identification of Peptide Having Melanogenesis Inhibitory Activity Using PS-SPCL

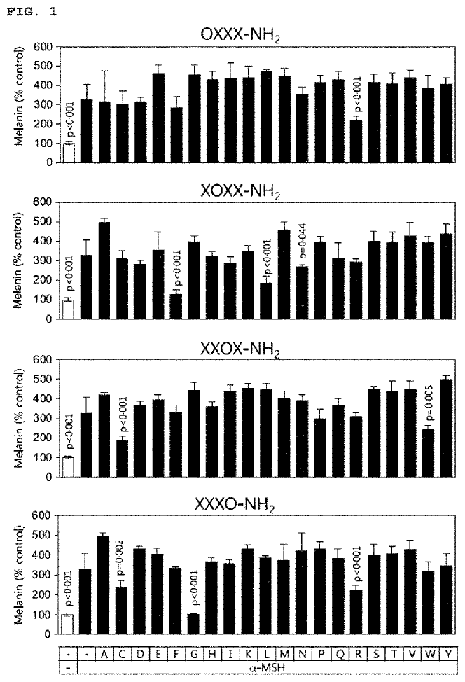

Screening using PS-SPCL was performed by treating B16-F10 melanoma cells, which had been stimulated with α-MSH, with all the peptide pools of synthetic tetrapeptide combinatorial library, and then observing an effect on melanogenesis (FIG. 1a).

As expected, α-MSH increased a melanin content of the B16-F10 cells, which was then weakened in various degrees by various tetrapeptide pools at a concentration of 1.0 mM. Out of those peptide pools, a tetrapeptide pool particularly having an excellent effect of inhibiting melanogenesis was confirmed. According to the above experimental results and an amino acid sequence of the identified peptide pool, a peptide pool having an arginine residue at a first position of an amino acid residue significantly inhibited melanogenesis in the cells stimulated with α-MSH. Similarly, peptide pools having phenylalanine or leucine at a second position, cysteine or tryptophan at a third position, and glycine, arginine or cysteine at a fourth position showed a significant effect of inhibiting melanin synthesis. Thus, a sequence of tetrapeptide having an anti-melanogenesis effect was predicted as follows according to positional scanning results: (Arg)-(Phe/Leu)-(Cys/Trp)-(Gly/Arg/Cys)-NH₂ (SEQ ID NO: 21).

### Example 2: Melanogenesis Inhibitory Effect of Individual Tetrapeptide

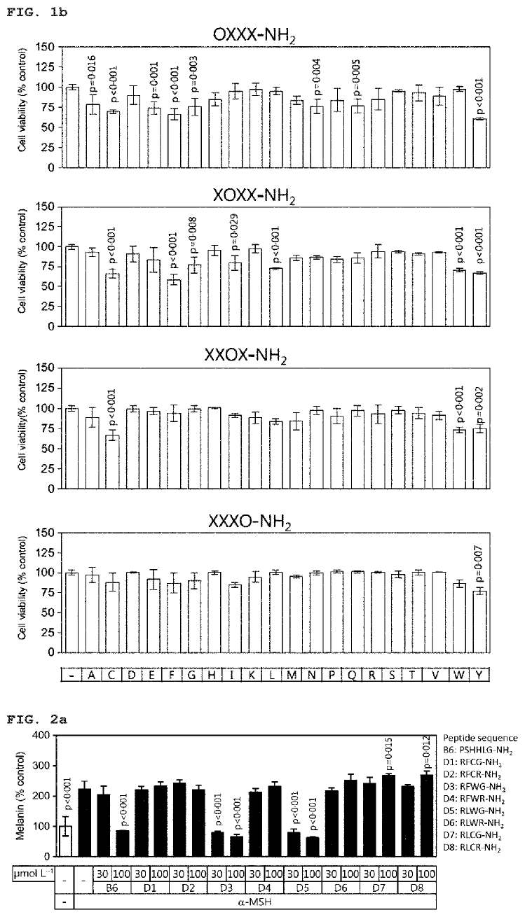

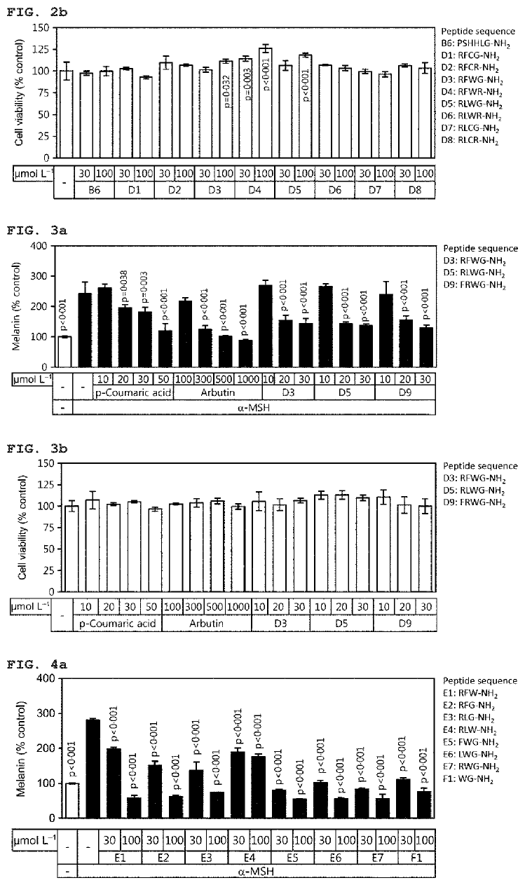

According to PS-SPCL results of the preceding example, eight individual tetrapeptides having a certain amino acid at each position of amino acid sequence were synthesized (FIGS. 2a and 3a).

These tetrapeptides include Arg at a first position; Phe or Leu at a second position; Cys or Trp at a third position; and Gly or Arg at a fourth position from an amino terminus, and were named as D1 to D8 (D1 peptide, RFCG-NH₂, SEQ ID No: 1; D2 peptide, RFCR—NH₂, SEQ ID NO: 2; D3 peptide, RFWG-NH₂, SEQ ID NO: 3; D4 peptide, RFWR—NH₂, SEQ ID NO: 4; D5 peptide, RLWG-NH₂, SEQ ID NO: 5; D6 peptide, RLWR-NH₂, SEQ ID NO: 6; D7 peptide, RLCG-NH₂, SEQ ID NO: 7; and D8 peptide, RLCR-NH₂, SEQ ID NO: 8). These individual peptides were evaluated for melanin inhibitory effect at concentrations of 30 μM and 100 μM. Hexapeptide B6 (Phe-Ser-His-His-Leu-Gly-NH₂) (SEQ ID NO: 20) was used as a control group. (Seok J K, Lee S W, Choi J, Kim Y M, Boo Y C. Identification of novel antimelanogenic hexapeptides via positional scanning of a synthetic peptide combinatorial library. Experimental Dermatology. 2017 August; 26(8):742-744). As shown in FIG. 2a, tetrapeptide D3 (Arg-Phe-Trp-Gly-NH₂) (SEQ ID NO: 2) and D5 (Arg-Leu-Trp-Gly-NH₂) (SEQ ID NO: 4) excellently inhibited melanogenesis in the cells, which were stimulated with α-MSH. Hexa-peptide B6 showed a melanogenesis inhibitory effect at 100 μM only.

The present inventors have found that a sequence of tetrapeptides D3 (Arg-Phe-Trp-Gly-NH₂) (SEQ ID NO: 2) and D5 (Arg-Leu-Trp-Gly-NH₂) (SEQ ID NO: 4) is similar to a sequence of α-MSH, that is, a part of Ac-SYSMEHFRWGKPV-NH₂ (SEQ ID NO: 19), which is FRWG, and thus included tetrapeptide D9 (FRWG-NH₂, SEQ ID NO: 9) in the following experiment. The present inventors have further confirmed anti-melanogenesis activity with regard to D3, D5 and D9 peptides according to the same method by using coumaric acid and arbutin as a positive control. When making a comparison based on a molar concentration, it was confirmed that not only tetrapeptides D3 and D5 but also tetrapeptide D9 have melanogenesis inhibitory activity at a concentration lower than that of p-coumaric acid and arbutin (See FIG. 3a).

### Example 3: Melanogenesis Inhibitory Effect of Short Peptide

In the preceding example, it was confirmed that a peptide having an amino acid sequence of D3, D5 and D9 has the most powerful melanogenesis inhibitory effect. A melanogenesis inhibitory effect and applicability thereof were further examined with regard to a peptide having a sequence shorter than that of the above tetrapeptide, which was expected to be more advantageous in terms of convenience, economic efficiency and the like of peptide synthesis (FIG. 4a).

To that end, E1 to E8 peptides were further synthesized: E1 (RFW-NH₂, SEQ ID NO: 10); E2 (RFG-NH₂, SEQ ID NO: 11); E3 (RLG-NH₂, SEQ ID NO: 12); E4 (RLW-NH₂, SEQ ID NO: 13); E5 (FWG-NH₂, SEQ ID NO: 14); E6 (LWG-NH₂, SEQ ID NO: 15); E7 (RWG-NH₂, SEQ ID NO: 16); and F1 (WG-NH₂, SEQ ID NO: 17). In case of the above E1 to F1 peptides, a carboxy terminus was amidated according to a chemical synthesis method, and an effect on melanogenesis was evaluated at a concentration of 30 μM to 100 μM.

As shown in FIG. 4a, the above peptides for additional analysis all showed an excellent melanin inhibitory effect. Out of the tested tripeptides, E5 (Phe-Trp-Gly-NH₂) (SEQ ID NO: 14), E6 (Leu-Trp-Gly-NH₂) (SEQ ID NO: 15) and E7 (Arg-Trp-Gly-NH₂) (SEQ ID NO: 16) containing Trp-Gly-NH₂ showed melanogenesis inhibitory activity higher than that of other tripeptides. Further, it was confirmed that dipeptide F1 (Trp-Gly-NH₂ (SEQ ID NO: 17), tryptophanyl glycinamide) also has an effect of melanogenesis inhibitory activity.

### Example 4: Melanogenesis Inhibitory Effect of Glycine Derivatives

The present inventors have further confirmed that glycinamide (Gly-NH₂, SEQ ID NO: 18, G1 peptide) maintains anti-melanogenesis activity, while free glycine and acetyl glycinamide have no activity.

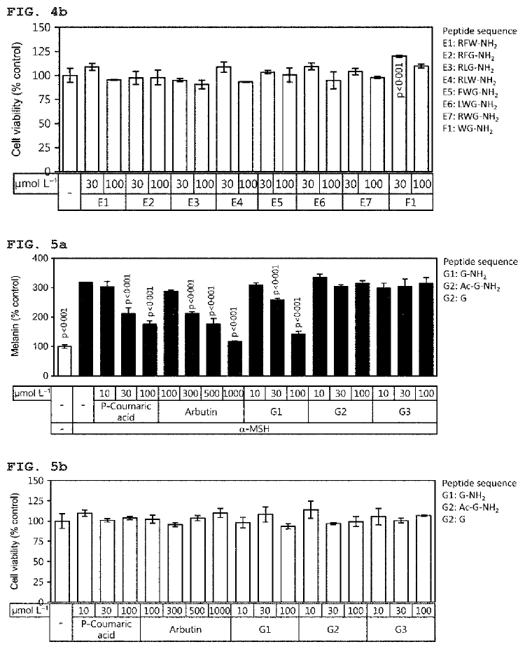

As shown in FIG. 5a, when making a comparison based on a molar concentration, it was assumed that a melanogenesis inhibitory effect of glycinamide is similar to that of p-coumaric acid and remarkably more excellent than that of arbutin.

### Example 5: Melanogenesis Inhibitory Effect on HEM Cell Line

To more clearly confirm experimental results of B16-F10 cells, an additional analysis was performed by using HEM cell lines (human epidermal melanocyte cells).

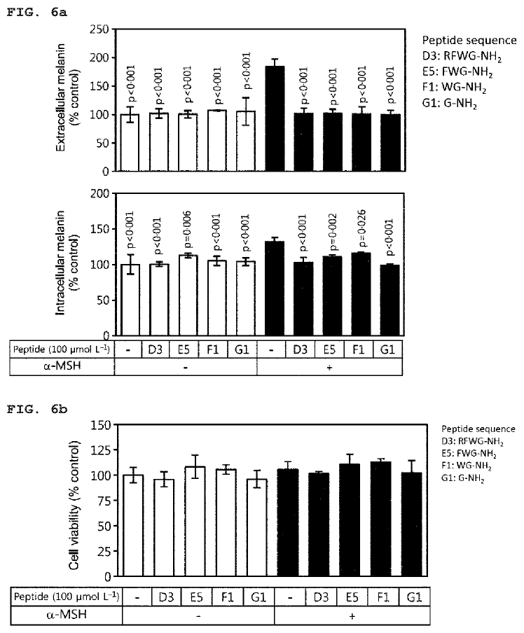

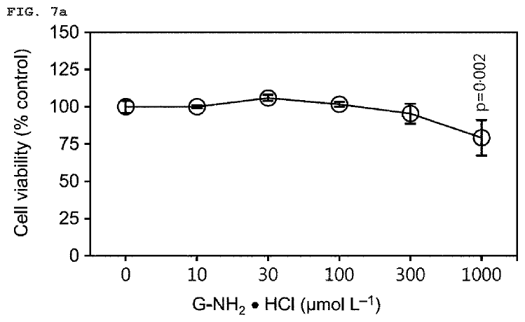

As shown in FIG. 6a, a content of intracellular and extracellular melanins in HEMs was increased by α-MSH treatment, and an increase in the melanin content was weakened by tetrapeptide D3, tripeptide E5, dipeptide F1 and monopeptide G1.

HEMs cell viability was not affected by peptide treatment (FIG. 6b).

Thus, it was clearly confirmed that the peptide of the present invention is a melanogenesis inhibitor which is safely available without cytotoxicity.

### Example 6: Efficacy Test of Glycinamide-HCl

### Example 6-1: Cytotoxicity and Melanogenesis Inhibitory Activity of Glycinamide-HCl

Cytotoxicity and melanogenesis inhibitory activity of glycinamide-HCl were more specifically confirmed by using glycinamide-HCl, which was an edible type of salt, while showing an effect of glycinamide.

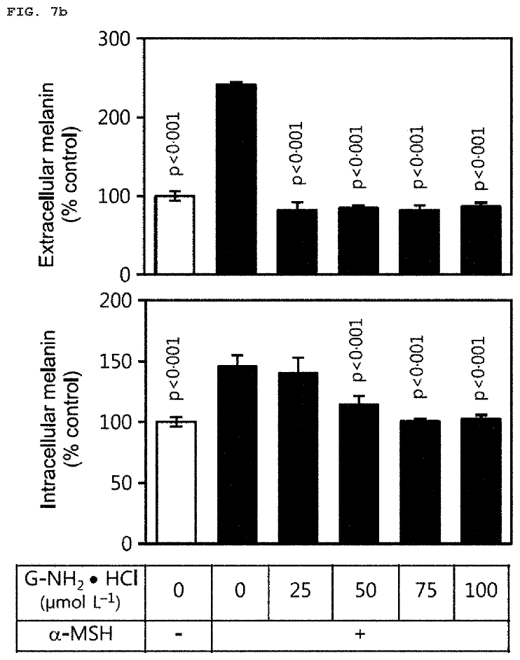

As shown in FIG. 7, glycinamide-HCl did not cause any cytotoxicity in the B16-F10 cells up to 300 μM. It was confirmed that glycinamide-HCl reduced a level of intracellular and extracellular melenins in the cells stimulated with α-MSH at 25-100 μM.

### Example 6-2: Measurement of TRY Activity and Related Protein Levels of Glycinamide-HCl

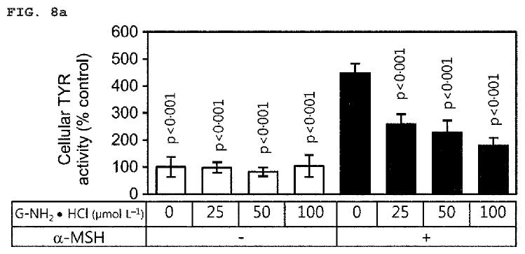

As shown in FIG. 8a, it was confirmed that α-MSH increased TYR activity and this change was significantly weakened by glycinamide-HCl. Further, α-MSH increased a protein level of tyrosinase (TYR) and tyrosinase-related protein 1 (TYRP1), but did not increase a protein expression level of dopachrometautomerase (DCT) or β-actin. However, in case of being treated with glycinamide-HCl, a protein level of TYR, TYRP1 and DCT was significantly reduced both in the presence and in the absence of α-MSH.

It was confirmed that glycinamide-HCl controls an expression level of enzymes related to melanin biosynthesis such as tyrosinase (TYR), tyrosinase-related protein 1 (TYRP1), dopachrome tautomerase (DCT, also called tyrosinase-related protein 2 (TYRP2)), etc., thus effectively inhibiting melanogenesis.

### Example 6-3: Effect of Glycinamide-HCl on CREB, ERK Phosphorylation and MITF Protein Expression Levels

It is known that an α-MSH-MC1R path induces a TYR expression by activating CREB and MITF. Thus, it was examined whether glycinamide-HCl affects a signaling path or not.

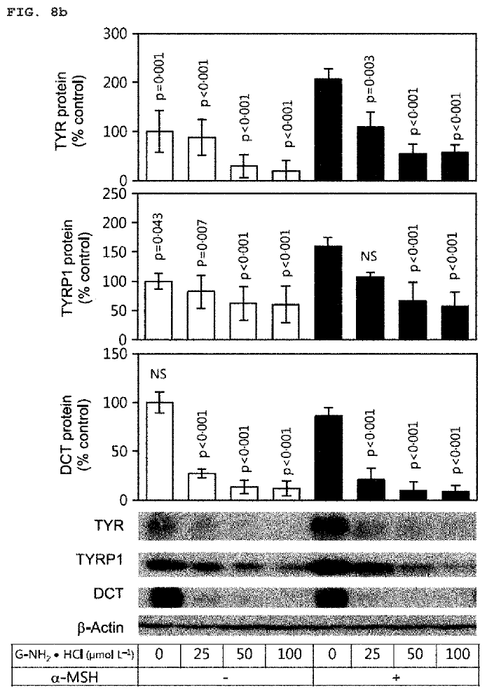

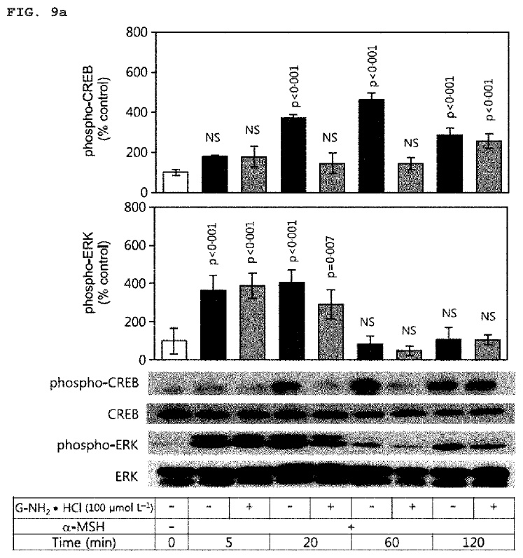

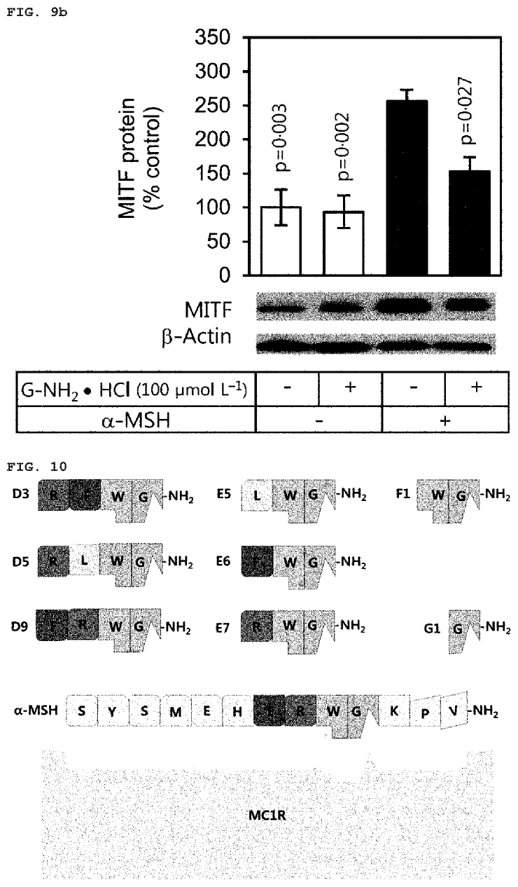

As shown in FIG. 9a, phosphorylation of CREB (Ser) was significantly increased in just 20 minutes after stimulation with α-MSH, and reached a maximum level in 60 minutes later as measured by western blotting. In the meantime, a total CREB value was not changed. α-MSH also increased phosphorylation of ERK (Thr and Tyr), but any significant change was not observed at a level of total ERK. Glycinamide-HCl inhibited phosphorylation of CREB, which had been stimulated with α-MSH, but did not affect phosphorylation of ERK.

Further, as shown in FIG. 9b, a-MSH increased a level of MITF expression, but was weakened by glycinamide-HCl.

Thus, it might be confirmed that glycinamide-HCl inhibits phosphorylation of CREB and ERK to reduce a protein expression of MITF, thereby effectively inhibiting melanogenesis.

### Example 7: Measurement of Cell Viability

After the B16-F10 cells and HEM were treated with the peptides of Examples 1 to 4 according to the same method, cell viability was measured and the results thereof were summarized in FIGS. 1b, 2b, 3b, 4b, 5b, 6b and 7a respectively.

In case of being treating with the peptide pool of Example 1 at a concentration of 1.0 mM, cell viability thereof was somewhat decreased depending on the peptide pool in some cases. However, it was determined that a melanogenesis inhibitory effect of a selected peptide pool is not attributable to a decrease in cell viability (FIG. 1b).

Out of individual peptides of Examples 2 to 4, some individual peptides somewhat decreased cell viability in some cases. However, it was also determined that a melanogenesis inhibitory effect of a selected peptide pool is not attributable to a decrease in cell viability. (FIGS. 2b to 5b) In addition, it was confirmed that D3, D5, F1 and G1 peptides have almost no cytotoxicity to HEM cell lines, which were additionally experimented (FIG. 6b).

Thus, it was confirmed that the peptides according to the present invention may be safely and effectively used due to low cytotoxicity.

### INDUSTRIAL APPLICABILITY

As described above, the peptide having melanogenesis inhibitory activity, which has been identified in the present invention, may be effectively used in developing medicine and medical supplies, cosmetic materials or functional foods for more safely and effectively preventing or treating pigmentation diseases.

1. A peptide with amidated (—NH₂) carboxy terminus comprising one amino acid sequence selected from the group consisting of SEQ ID NO: 3, SEQ ID NO: 5 and SEQ ID NO: 9 to 18.

2. The peptide of claim 1, wherein the peptide includes one amino acid sequence selected from the group consisting of SEQ ID NO: 14 to 18.

3. The peptide of claim 1, wherein the peptide has melanogenesis inhibitory activity.

4. A pharmaceutical composition for preventing or treating pigmentation diseases comprising at least one peptide with amidated (—NH₂) carboxy terminus selected from the group consisting of SEQ ID NO: 3, SEQ ID NO: 5 and SEQ ID NO: 9 to 18 as an active ingredient.

5. A cosmetic composition comprising at least one peptide with amidated (—NH₂) carboxy terminus selected from the group consisting of SEQ ID NO: 3, SEQ ID NO: 5 and SEQ ID NO: 9 to 18.

6. A food composition comprising at least one peptide with amidated (—NH₂) carboxy terminus selected from the group consisting of SEQ ID NO: 3, SEQ ID NO: 5 and SEQ ID NO: 9 to 18.

7. The pharmaceutical composition of claim 4, wherein the pigmentation diseases are at least one selected from the group consisting of melasma, freckles, dark circles, lentigos, age spots, melanocytic nevus, cafe au lait spots, Ota nevus, blue nevus, hyperpigmented nevus, pigmentation of nipples, pigmentation of labia minora and blemishes.

8. The composition of claim 5, wherein the composition is used for whitening.

9. The composition of claim 6, wherein the composition includes one form selected from the group consisting of a functional food, a nutritional supplement, a health functional food and food additives.

10. A use of at least one peptide with amidated (—NH₂) carboxy terminus for preparing a formulation for preventing or treating pigmentation diseases selected from the group consisting of SEQ ID NO: 3, SEQ ID NO: 5 and SEQ ID NO: 9 to 18.

11. A method for treating pigmentation diseases, the method comprising: administering an effective amount of a composition containing at least one peptide with amidated (—NH₂) carboxy terminus selected from the group consisting of SEQ ID NO: 3, SEQ ID NO: 5 and SEQ ID NO: 9 to 18 into a subject in need thereof.

12. The composition of claim 6, wherein the composition is used for whitening.


SEQUENCE LISTING (21 sequences)

SEQ ID NO: 1 - protein, 4 aa, Artificial Sequence
  D1 peptide
RFCG

SEQ ID NO: 2 - protein, 4 aa, Artificial Sequence
  D2 peptide
RFCR

SEQ ID NO: 3 - protein, 4 aa, Artificial Sequence
  D3 peptide
RFWG

SEQ ID NO: 4 - protein, 4 aa, Artificial Sequence
  D4 peptide
RFWR

SEQ ID NO: 5 - protein, 4 aa, Artificial Sequence
  D5 peptide
RLWG

SEQ ID NO: 6 - protein, 4 aa, Artificial Sequence
  D6 peptide
RLWR

SEQ ID NO: 7 - protein, 4 aa, Artificial Sequence
  D7 peptide
RLCG

SEQ ID NO: 8 - protein, 4 aa, Artificial Sequence
  D8 peptide
RLCR

SEQ ID NO: 9 - protein, 4 aa, Artificial Sequence
  D9 peptide
FRWG

SEQ ID NO: 10 - protein, 3 aa, Artificial Sequence
  E1 peptide
RFW

SEQ ID NO: 11 - protein, 3 aa, Artificial Sequence
  E2 peptide
RFG

SEQ ID NO: 12 - protein, 3 aa, Artificial Sequence
  E3 peptide
RLG

SEQ ID NO: 13 - protein, 3 aa, Artificial Sequence
  E4 peptide
RLW

SEQ ID NO: 14 - protein, 3 aa, Artificial Sequence
  E5 peptide
FWG

SEQ ID NO: 15 - protein, 3 aa, Artificial Sequence
  E6 peptide
LWG

SEQ ID NO: 16 - protein, 3 aa, Artificial Sequence
  E7 peptide
RWG

SEQ ID NO: 17 - protein, 2 aa, Artificial Sequence
  F1 peptide
WG

SEQ ID NO: 18 - protein, 1 aa, Artificial Sequence
  G1 peptide
G

SEQ ID NO: 19 - protein, 13 aa, Artificial Sequence
  alpha-melanocyte stimulating hormone
SYSMEHFRWGKPV

SEQ ID NO: 20 - protein, 6 aa, Artificial Sequence
  B6 hexapeptide
FSHHLG

SEQ ID NO: 21 - protein, 4 aa, Artificial Sequence
  tetrapeptide
  VARIANT (2): Xaa is Phe or Leu
  VARIANT (3): Xaa is Cys or Trp
  VARIANT (4): Xaa is Gly, Arg or Cys
RXXX